(19) United States
(12) Patent Application Publication
(10) Pub. No.: US 2002/0016314 A1
(43) Pub. Date: Feb. 7, 2002

(54) Compositions containing phytosterol and policosanol esters of fatty acids for reducing blood cholesterol and triglycerides

(76) Inventor: Endre Markovits Schersl, Quilpue (CL)

Correspondence Address: David I. ROCHE, BAKER & McKENZIE, 130 E. Randolph Drive, Chicago, IL, 60601, US

(21) Appl. No.: 09/772,790

(22) Filed: Jan. 30, 2001

(30) Foreign Application Priority Data
Jan. 31, 2000 (CL) 209-2000

(51) Int. Cl. A61K 31/56; A61K 47/00; A23D 7/005

(52) U.S. Cl. 514/169; 424/439; 426/601

(57) ABSTRACT

A composition for lowering LDL-cholesterol levels or for elevating HDL-cholesterol levels in blood of a mammal or both, comprised of an ester of a policosanol or a mixture or esters of policosanols and a method for lowering LDL-cholesterol levels or for elevating HDL-cholesterol levels in blood of a mammal or both, comprised of orally administering to said mammal a composition comprising an effective amount of an ester of a policosanol or a mixture of esters of policosanols.

A composition for lowering LDL-cholesterol and triglycerides or for elevating HDL-cholesterol in blood of a mammal or both, comprised of an ester of a phytosterol or a mixture of esters of phytosterols wherein the acid moiety of the ester or the mixture of esters is fatty acid selected from the group consisting of eicosapentaenoic acid, docosapentaenoic acid, linoleic acid, linolenic acid and arachidonic acid or a mixture of said esters, and a method for lowering LDL-cholesterol and triglycerides or for elevating HDL-cholesterol in blood of a mammal or both, comprised of orally administering to said mammal a composition comprising an effective amount of an ester of a phytosterol or a mixture of esters of a phytosterols wherein the acid moiety of the ester or the mixture esters is a fatty acid selected from the group consisting of eicosapentaenoic acid, docosapentaenoic acid, linoleic acid, linolenic acid and arachidonic acid.

A second composition for lowering LDL-cholesterol and triglycerides or for elevating HDL-cholesterol in blood of a mammal or both, comprised of an ester of a policosanol or a mixture of esters of policosanol and an ester of a phytosterol or a mixture of esters of phytosterols wherein the acid moiety of the ester of the phytosterol or the mixture of esters of the phytosterols is a fatty acid and a second method for lowering LDL-cholesterol and triglycerides or for elevating HDL-cholesterol in blood of a mammal or both, comprised of orally administrating to said mammal a composition containing an effective amount of an ester of a policosanol or a mixture of esters of policosanols, and an ester of a phytosterol or a mixture of esters of phytosterols wherein the acid moiety of the ester of the phytosterol and the mixture of esters of the phytosterols is a fatty acid.

### BACKGROUND OF THE INVENTION

The present invention is related to food and pharmaceutical compositions and methods suitable for lowering cholesterol and triglyceride levels or for elevating HDL-cholesterol levels in the blood of a mammal, particularly compositions containing phytosterol esters of omega-3 and omega-6 polyunsaturated fatty acids and policosanols esters.

Disorders of lipid metabolism, especially the harmful effects caused by high cholesterol and triglyceride levels in the blood, have been intensively studied for many decades.

Cholesterol levels in the blood over 200 mg/dl constitute the main risk factor of coronary diseases, the most frequent cause of death, principally in developed countries. However, the risk factor is not only related to a high cholesterol level in blood, but also to the different forms of total cholesterol. A high level of low-density lipoprotein, or LDL-cholesterol, and very low-density lipoprotein, or VLDL-cholesterol, in blood constitutes a problem because these lipoproteins are very likely to remain in the cardiovascular system causing the formation of plaques in the coronary arteries. Likewise, low levels of high-density lipoproteins, or HDL-cholesterol, constitute an additional risk factor because they are useful in removing the form of cholesterol that blocks arteries. Therefore total cholesterol levels and total cholesterol/HDL-cholesterol ratios must be considered for evaluating the risk of coronary diseases.

However, not only cholesterol, but also high triglyceride levels in blood constitute a risk factor of coronary diseases and other complications (PUFA NEWSLETTER, vol.2, June 1998).

In general, the treatment of lipid metabolism disorders has been mostly addressed to treating hypercholesterolemia using different food and pharmaceutical compositions that lower elevated cholesterol level in blood. Many of these compositions contain plant sterols or phytosterols which would interfere or obstruct the intestinal absorption of dietary cholesterol and reduce LDL-cholesterol. There is a vast scientific production related to this subject which is reviewed in U.S. Pat. No. 5,958,913, quoting over 70 references concerning the effects and mechanisms of dietary phytosterols on the reduction of blood cholesterol.

U.S. Pat. No. 5,244,887 discloses a method for the elaboration of a composition to be used as a food additive which contains one or more stanols, a solubilizing agent, an antioxidant and a dispersing agent. The stanols are obtained by catalytic hydrogenation of sterols. These food compositions are intended for reducing cholesterol absorption from foods.

U.S. Pat. No. 5,932,652 discloses a water dispersive food composition for reducing cholesterol absorption containing sitostanol (beta-sitostanol) and lecithin.

In order to increase the inhibition of dietary cholesterol absorption, U.S. Pat. No. 5,591,836 discloses a method that uses a saponin compound containing 5-C-hydroxymethylhexose and sterol or terpene.

U.S. Pat. No. 5,747,464 discloses the utilization of complexes formed by beta-sitostanol and pectin. Sterols esterified with fatty acids seem to be more efficient cholesterol absorption inhibitors than free sterols.

U.S. Pat. No. 5,958,913 discloses the utilization of stanol esters, mainly the fatty acid ester of beta-sitostanol, where the fatty acids are derived from raps seed oil. This patent also presents long clinical studies on the efficiency of these esters for inhibiting intestinal absorption of cholesterol and the lowering LDL-cholesterol in the blood.

Long chain lineal saturated primary alcohols from 20 to 38 carbon atoms, also called fatty alcohols or higher aliphatic alcohols, also known as policosanols, are efficient to reduce blood cholesterol.

In the present invention the term “policosanol” is used as meaning a lineal saturated primary alcohol containing 20 or more carbon atoms. The mechanism of action of policosanols is not known with certainty, but it is believed they would affect synthesis of cholesterol in the liver. A considerable reduction of total cholesterol levels and LDL-cholesterol levels in the blood of patients with diabetes mellitus upon sustained ingestion of small amounts of policosanols have been observed (Omayda Torres et al., Diabetes Care, “Treatment of Hypercholesterolemia in NIDDM with Polycosanol”, 1995, vol. 18, No 5, 393-396).

U.S. Pat. No. 5,856,316 discloses a process for obtaining policosanols from sugarcane wax and their utilization in the treatment of hypercholesterolemia. Policosanols from sugarcane wax comprise a mixture of aliphatic alcohols from 24 to 34 carbon atoms and they were effective hypocholesterolemic agents administered in daily doses from 1 to 100 mg.

U.S. Pat. No. 5,952,893 discloses a composition for reducing cholesterol levels in the blood comprising a mixture of phytosterols (mixture of different plant sterols) and policosanols with a synergistic effect. Phytosterols of the invention comprise beta-sitosterol, campesterol and stigmasterol derived from vegetable oils and the policosanols of the invention comprise a mixture of fatty alcohols containing from 22 to 36 carbon atoms derived from rice bran wax. These policosanols are commercially available (“Rice Bran Wax”, Traco Labs Inc.). However, free phytosterols and free policosanols are barely soluble in the micelle phase of food channels, therefore its efficiency to reduce blood cholesterol is rather low, which leads to the necessity of using relatively high doses of these compounds.

In addition, food and pharmaceutical compositions containing free phytosterols and/or free policosanols are not effective for reducing triglyceride levels in blood.

Accordingly, an objective of the present invention is to provide food and pharmaceutical compositions for lowering LDL-cholesterol levels or for elevating HDL-cholesterol levels in the blood of a mammal or both, said compositions containing easily absorbable forms of policosanols in the digestive tract of said mammal, said easily absorbable forms of policosanols comprising an ester of a policosanol and a carboxilic acid countering from 2 to 22 carbon atoms, denoted simply as a policosanol ester.

A further objective of the present invention is to provide a method for lowering LDL-cholesterol or for elevating HDL-cholesterol in the blood of a mammal or both, by administering orally to said mammal food or pharmaceutical compositions containing an effective amount of a policosanol ester or mixture of policosanol esters wherein the acid moiety of the esters is an carboxilic acid containing from 2 to 22 carbon atoms.

A further objective of the present invention is to provide food or pharmaceutical compositions for lowering LDL-cholesterol and triglycerides levels or for elevating HDL-cholesterol levels in the blood of a mammal or both. Said compositions comprise an ester of a phytosterols and an omega-3 long chain polyunsaturated fatty acids such as eicosapentaeinoic acid (EPA), docosahexaenoic acid (DHA), linolenic acid or an ester of a phytosterol and an omega-6 long chain polyunsaturated fatty acid such as linoleic acid or araquidonic acid, or a mixture of said esters.

A further objective of the present invention is to provide a method for lowering LDL-cholesterol and triglycerides levels or for elevating HDL-cholesterol levels in the blood of a mammal or both, by administering orally to said mammal food or pharmaceutical compositions containing an effective amount of an ester of a phytosterols, preferably beta-sitosterol or beta-sitostanol, and an omega-3 long chain polyunsaturated fatty acids such as eicosapentaeinoic acid (EPA), docosahexaenoic acid (DHA), linolenic acid or an ester of a phytosterol and an omega-6 long chain polyunsaturated fatty acid such as linoleic acid or araquidonic acid, or a mixture of said esters.

The objective of providing food or pharmaceutical compositions for lowering LDL-cholesterol and triglycerides levels or raising HDL-cholesterol levels in the blood of a mammal or both may be achieved also by means of a composition comprising mixtures formed by one or more policosanols esters and one or more esters of a phytosterols and an omega-3 long chain polyunsaturated fatty acids such as eicosapentaeinoic acid (EPA), docosahexaenoic acid (DHA), linolenic acid or an ester of a phytosterol and an omega-6 long chain polyunsaturated fatty acid such as linoleic acid or araquidonic acid.

The objective of providing a method for lowering LDL-cholesterol and triglycerides levels or raising HDL-cholesterol levels in the blood of a mammal or both, may be achieved also by orally administering to said mammal food or pharmaceutical compositions containing an effective amount of a mixture formed by one or more policosanols esters and one or more esters of a phytosterol, preferably beta-sitosterol or beta-sitostanol, and an omega-3 long chain polyunsaturated fatty acids such as eicosapentaeinoic acid (EPA), docosahexaenoic acid (DHA), linolenic acid or an ester of a phytosterol and an omega-6 long chain polyunsaturated fatty acid such as linoleic acid or araquidonic acid.

In accordance with the present invention a composition for lowering LDL-cholesterol levels or for elevating HDL-cholesterol levels in the blood of a mammal or both, comprises an ester of a policosanol or a mixture or esters of policosanols and a method for lowering LDL-cholesterol levels or for elevating HDL-cholesterol levels in the blood of a mammal or both, comprises orally administering to said mammal a composition comprising an effective amount of an ester of a policosanol or a mixture of esters of policosanols.

Also according with the present invention a composition for lowering LDL-cholesterol and triglycerides or for elevating HDL-cholesterol in the blood of a mammal or both, comprises an ester of a phytosterol or a mixture of esters of phytosterols wherein the acid moiety of the ester or the mixture of esters is fatty acid selected from the group consisting of eicosapentaenoic acid, docosapentaenoic acid, linoleic acid, linolenic acid and arachidonic acid or a mixture of said esters and a method for lowering LDL-cholesterol and triglycerides or for elevating HDL-cholesterol in the blood of a mammal or both, comprises orally administering to said mammal a composition comprising an effective amount of an ester of a phytosterol or a mixture of esters of a phytosterols wherein the acid moiety of the ester or the mixture esters is a fatty acid selected from the group consisting of eicosapentaenoic acid, docosapentaenoic acid, linoleic acid, linolenic acid and arachidonic acid.

A second composition for lowering LDL-cholesterol and triglycerides or for elevating HDL-cholesterol in the blood of a mammal or both, in accordance with the present invention, comprises an ester of a policosanol or a mixture of esters of policosanol and an ester of a phytosterol or a mixture of esters of phytosterols wherein the acid moiety of the ester of the phytosterol or the mixture of esters of the phytosterols is a fatty acid and a second method for lowering LDL-cholesterol and triglycerides or for elevating HDL-cholesterol in the blood of a mammal or both in accordance with the present invention comprises orally administrating to said mammal a composition containing an effective amount of an ester of a policosanol or a mixture of esters of policosanols, and an ester of a phytosterol or a mixture of esters of phytosterols wherein the acid moiety of the ester of the phytosterol and the mixture of esters of the phytosterols is a fatty acid.

Policosanol ester utilized in the present invention were prepared by transesterification of a mixture containing policosanols and a mixture containing ethyl or methyl esters of fatty acids using sodium ethylate as catalyst.

Policosanols from 20 to 26 carbon atoms can be obtained from the neutral fraction of tall oil as described in Chilean Patent Application No 873/98. Other sources such as sugarcane wax, rice bran wax are suitable to the purposes of this invention. Table I shows the average composition of policosanols in Tall Oil, Rice Bran Wax and Sugarcane Wax.

From Table I it is possible to observe that the three sources do not provide a complete range of 20-to-36 carbon atom policosanols separately, but they do together.

TABLE I
Relative composition of fatty alcohols
obtained from different sources

| Policosanol | Tall oil | Rice bran wax | Sugarcane wax |
|---|---|---|---|
| Eicosanol C20 | 0.2 | — | — |
| Heneicosanol C21 | 0.1 | — | — |
| Docosanol C22 | 50.7 | 1.1 | — |
| Tricosanol C23 | 2.7 | — | — |
| Tetracosanol C24 | 45.0 | 11.6 | 0.7 |
| Pentacosanol C25 | 0.3 | — | — |
| Hexacosanol C26 | 1.0 | 10.6 | 8.0 |
| Heptacosanol C27 | — | — | 3.5 |
| Octacosanol C28 | — | 20.2 | 66.0 |
| Nonacosanol C29 | — | — | 0.8 |
| Triacontanol C30 | — | 30.1 | 13.5 |
| Dotriacontanol C32 | — | 16.8 | 6.0 |
| Tetratriacontanol C34 | — | 8.0 | 1.5 |
| Hexatriacontanol C36 | — | 1.4 | — |

The ethyl or methyl esters of fatty acids of the present invention are obtained from vegetable or animal oils by methods well known in the state of the art. These techniques comprise saponifying of oil followed by the separation of glycerol and soaps resulting from the saponifying process. Soaps are acidulated and then transformed into fatty acids and these fatty acids esterified with methanol or ethanol using sulfuric acid as catalyst.

In the present invention, the process of production of policosanol esters is carried out in a solvent free process. Therefore these esters, which have good miscibility with fats and oils, can be safely incorporated into different fatty foods such as edible oil, margarine, mayonnaise, sauces, or milk. Thus, an objective of the present invention is achieved providing a food composition containing forms of policosanol easily absorbable in the digestive tract of a mammal, suitable for lowering LDL-cholesterol levels or for elevating HDL-cholesterol levels in the blood or both of said mammal. These easily absorbable forms of policosanol are the polycosanol esters of the present invention which, when incorporated into some suitable food substance such as table margarine, shortening, ice cream, yogurt and others, form food compositions suitable for lowering LDL-cholesterol levels or for elevating HDL-cholesterol levels in the blood or both of a mammal, upon ingestion by said mammal of an effective amount of the food composition.

Likewise, polycosanol esters can be incorporated into pharmaceutical compositions in the form of capsules. These capsules may also comprise a pharmaceutically acceptable component such as an excipient, diluent, antioxidant, coloring agent and stabilizer. Pharmaceutical composition can also be provided in the form of tablets containing policosanol esters which may also comprise a pharmaceutically acceptable component, such as an excipient, coloring agent, antioxidant, binder and stabilizer. Said tablets and capsules form pharmaceutical compositions suitable for lowering LDL-cholesterol levels or for elevating HDL-cholesterol levels in the blood or both of a mammal, upon ingestion by said mammal of an effective amount of the pharmaceutical composition.

A further objective is to provide food or pharmaceutical compositions suitable for lowering LDL-cholesterol and triglyceride levels, or for elevating HDL-cholesterol levels, in the blood of a mammal or both, can be achieved esterifying a phytosterol with an omega-3 or omega-6 long chain polyunsaturated fatty acid and incorporating said esters into some suitable food substance such as table margarine, shortening, ice cream, yogurt and others, or in pharmaceutical forms such as tablets or capsules or both which may also comprise a pharmaceutically acceptable component such as an excipient, coloring agent, antioxidant, binder and stabilizer.

Still a further objective of the present invention is to provide a method for lowering LDL-cholesterol and triglyceride levels or for elevating HDL-cholesterol levels in the blood of a mammal or both, is achieved by administering orally to said mammal an effective amount of food or pharmaceutical composition comprising a phytosterol, preferably beta-sitosterol or beta-sitostanol, with an omega-3 or omega-6 long chain polyunsaturated fatty acid ester, said esters incorporated into some suitable food substance such as table margarine, shortening, ice cream, yogurt and others, or in a pharmaceutical form such as tablets or capsules or both, which may also comprise a pharmaceutically acceptable component such as an excipient, coloring agent, antioxidant, binder and stabilizer.

Food and pharmaceutical compositions suitable for lowering LDL-cholesterol and triglyceride levels or for elevating HDL-cholesterol levels in the blood of a mammal or both, can also be provided by incorporating one or more policosanols esters and one or more esters of a phytosterol and an omega-3 or omega-6 long chain polyunsaturated fatty acid into some suitable food substance, such as table margarine, shortening, ice cream, yogurt and others, or in pharmaceutical forms such as tablets or capsules or both which may also comprise a pharmaceutically acceptable component such as an excipient, coloring agent, antioxidant, binder and stabilizer.

The method of lowering LDL-cholesterol and triglyceride levels or for elevating HDL-cholesterol levels in the blood of a mammal or both, may also be achieved by administering orally to said mammal an effective amount of food or pharmaceutical composition comprising one or more policosanol ester and one or more esters of a phytosterol and an omega-3 or omega-6 long chain polyunsaturated fatty acid incorporated into some suitable food substance, such as table margarine, shortening, ice cream, yogurt and others, or in pharmaceutical forms such as tablets or capsules or both which may also comprise a pharmaceutically acceptable component such as an excipient, coloring agent, antioxidant, binder and stabilizer.

The following examples are presented in illustration of the compositions and methods of this invention and are not intended as an undue limitation on the generally broad scope thereof.

### EXAMPLE 1

### Preparation of Polycosanol Esters

104.3 g of ethyl-PUFA and 98.5 g of a mixture of policosanols were mixed in a 500-ml flask, the mixture were heated at the temperature of 180° C. and the pressure of 5 mbar for 120 minutes to remove air from the mixture. After breaking the vacuum with nitrogen, 2.5 g o sodium ethylate were added to the flask and the mixture was further heated at the reduced pressure for 24 hours. After breaking the vacuum with nitrogen and removing the reaction mixture this was mixed with hot water to remove the catalysts, the oily phase was separated and vacuum dried obtaining 103.1 g of polycosanol esters.

### EXAMPLE 2

### Preparation of a Food Composition With Policosanol Ester

A portion of polycosanol esters from Example 1 was mixed with corn oil (3% in weight of the mixture) and a mayonnaise with the following composition was prepared:

| Ingredient | % |
|---|---|
| % oil-policosanol mixture | 70.0 |
| Thickening agent | 1.5 |
| Salt | 1.0 |
| Sugar | 1.0 |
| Vinegar (4% in weight) | 6.0 |
| Water | 17.0 |
| Soy lecithin | 1.5 |
| Mustard | 2.0 |
| Total | 100.0 |

Mayonnaise was prepared using a home homogenizer. Its organoleptic properties did not differ from conventional mayonnaise.

### EXAMPLE 3

### Preparation of Phytosterol-PUFA

118.4 g of ethyl-PUFA and 140.0 g of a mixture of phytosterols were mixed in a 500-ml flask, the mixture were heated at the temperature of 95° C. and the pressure of 5 mbar for 120 minutes to remove air from the mixture. After breaking the vacuum with nitrogen, 4.2 g o sodium ethylate were added to the flask and the mixture was further heated at the reduced pressure for 24 hours. After breaking the vacuum with nitrogen and removing the reaction mixture this was mixed with hot water to remove the catalysts, the oily phase was separated and vacuum dried obtaining 156.3 g of phytosteryl-PUFA.

### EXAMPLE 4

### Preparation of a Food Composition With Phytosteryl-PUFA

A portion of phytosteryl-PUFA from Example 3 was mixed with lard. 1000 g of lard were melted at 100° C. in water bath and 10 g of phytosteryl-PUFA were incorporated. The lard was used for the elaboration of bread containing 20% of fatty matter with respect to flour used. The organoleptic characteristics of bread do not differ from conventional bread.

### EXAMPLE 5

### Short Term Nutritional Evaluation in Rats. Effect of Phytosteryl-PUFA on Serum and Hepatic Lipids in Rats

24 Sprague Dawley male rats divided into four groups of six animals each were fed for nine days with the following diet: the C0 group was fed with a basal food comprising Champion pellets ground and powdered and mixed with corn oil (3.3% in weight of the mixture). The C1 group was fed with mixture comprising basal and cholesterol (1% in weight of the mixture). The A1 group was fed with a mixture comprising basal food, 1% of cholesterol and 1% of stanol esters in weight of the mixture. Finally, the A2 group was fed with a mixture comprising the basal food, 1% of cholesterol and 1% of phytosteryl-PUFA in weight of the mixture.

The stanol esters comprised a mixture of beta-sitostanol and campestanol esters of fatty acids obtained from rape seed oil. Phytosteryl-PUFA esters were prepared according to Example 3. Dietary treatment was individually applied and corporal weight and dietary ingestion were measured. After the nine days of feeding, total lipids and cholesterol in the liver and cholesterol and triglycerides in the blood serum of each animal were determined. Tables I and II show the results:

TABLE I
Total lipids and total cholesterol in the liver

| | Total lipids | Cholesterol |
|---|---|---|
| | (mg/g liver) | (mg/g liver) |
| C₀ | 34.99 ± 2.23 (5) | 1.53 ± 0.12 (5) |
| C₁ | 40.22 ± 0.99 (5) | 2.82 ± 0.19 (6) |
| A₁ | 30.66 ± 1.44 (6) | 1.28 ± 0.15 (5) |
| A₂ | 28.79 ± 1.48 (4) | 0.99 ± 0.004 (5) |

Figures represent mg/g of liver and the results are presented as an average per group±standard error of the sample. The number of samples analyzed appears in parentheses.

Pairwise comparison of the means using Student test, indicate that there is a significant difference between C1 and A1 or A2 in total lipids and total cholesterol at a significance level of 5% in both cases. Also, there is a significant difference between A1 and A2 in total lipids and total cholesterol at 10% and 5% level of significance respectively.

TABLE II
Total cholesterol and triglycerides in blood serum.

| | Total cholesterol | Triglycerides |
|---|---|---|
| C0 | 68.44 ± 7.13 (6) | 21.75 ± 2.16 (6) |
| C1 | 140.17 ± 7.80 (6) | 34.11 ± 3.36 (5) |
| A1 | 120.68 ± 11.14 (5) | 33.42 ± 6.26 (4) |
| A2 | 126.10 ± 3.81 (5) | 29.74 ± 4.13 (5) |

Figures represent mg/dl and the results are presented as an average per group±standard error of the sample. The number of analyzed samples appears in parentheses.

From the results it is possible to conclude that a significant difference exists between C1 and A1 or A2 in total serum cholesterol at a significance level of 1%, but the difference between A1 and A2, is not significant at a significance level of 10%. Concerning triglycerides, there is no significant difference with a significance level of 10% between C1 and A1 , but between the difference between C1 and A2 is significant at a significance level of 10%. Likewise, between A1 and A2, there is a significant difference with a significance level of 20%.

Blood serum levels of HDL-cholesterol in A1 and A2 were also measured and results are shown in Table III.

TABLE III
Serum levels of HDL cholesterol

| | HDL |
|---|---|
| A1 | 37.96 ± 1.97 (6) |
| A2 | 41.78 ± 1.65 (5) |

Figures represent mg/dl and results are presented as an average per group±standard error of the sample. The number of analyzed samples appears in parentheses.

HDL-cholesterol is higher in A2 group than in the A1 group with a significance level of 1%.

What is claimed is:

1. A composition for lowering LDL-cholesterol levels or for elevating HDL-cholesterol levels in blood of a mammal or both, comprising an ester of a policosanol or a mixture or esters of policosanols.

2. The composition according to claim 1, wherein the acid moiety of the ester and the esters is a carboxylic acid containing from 2 to 22 carbon atoms.

3. The composition according to claims 1 or 2, further comprising a food substance or a mixture of food substances.

4. The composition according to claim 3, wherein the food substance or mixture of food substances is selected from the group consisting of table margarine, shortening, mayonnaise, vegetable oil, ice cream, milk and yogurt.

5. The composition according to claim 1 or 2, further comprising a pharmaceutically acceptable component selected from the group consisting of an excipient, antioxidant, coloring agent, binder and stabilizer.

6. A method for lowering LDL-cholesterol levels or for elevating HDL-cholesterol levels in blood of a mammal or both, which comprises orally administering to said mammal a composition comprising an effective amount of an ester of a policosanol or a mixture of esters of plicosanols

7. The method according to claim 6, wherein the acid moiety of the ester and the esters comprise a carboxylic acid containing from 2 to 22 carbon atoms.

8. The method according to claims 7, wherein the composition further comprises a food substance or a mixture of food substances.

9. The method according to claim 8, wherein the food substance o the mixture of food substances is selected from the group consisting of table margarine, shortening, mayonnaise, vegetable oil, ice cream, milk and yogurt.

10. The method according to claim 7, wherein the composition further comprises a pharmaceutically acceptable component selected from the group consisting of an excipient, antioxidant, coloring agent, binder and stabilizer.

11. The method according to claim 9 or 10, wherein the effective amount of the ester of the policosanol or the mixture of the esters of the policosanols of the composition comprises a daily dosage from 1 to 500 mg of said ester or said mixture of esters.

12. A composition for lowering LDL-cholesterol and triglycerides or for elevating HDL-cholesterol in blood of a mammal or both, comprising an ester of a phytosterol or a mixture of esters of phytosterols wherein the acid moiety of the ester or the mixture of esters is fatty acid selected from the group consisting of eicosapentaenoic acid, docosapentaenoic acid, linoleic acid, linolenic acid and arachidonic acid or a mixture of said esters.

13. The composition according to claim 12, wherein the phytosterol is selected from the group consisting of beta-sitosterol, beta-sitostanol, campesterol, campestanol and stigmasterol.

14. The composition according to claims 12 or 13, further comprising a food substance or a mixture of food substances.

15. The composition according to claim 14, wherein the food substance o mixture of food substances is selected from the group consisting of table margarine, shortening, mayonnaise, vegetable oil, ice cream, milk and yogurt.

16. The composition according to claims 12 or 13 further comprising a pharmaceutically acceptable component selected from the group consisting of an excipient, antioxidant, coloring agent, binder and stabilizer.

17. A method for lowering LDL-cholesterol and triglycerides or for elevating HDL-cholesterol in blood of a mammal or both, which comprises orally administering to said mammal a composition comprising an effective amount of an ester of a phytosterol or a mixture of esters of a phytosterols wherein the acid moiety of the ester or the mixture esters is a fatty acid selected from the group consisting of eicosapentaenoic acid, docosapentaenoic acid, linoleic acid, linolenic acid and arachidonic acid.

18. The method according to claim 17, wherein the phytosterol is selected from the group consisting of beta-sitosterol, beta-sitostanol, campesterol, campestanol and stigmasterol.

19. The method according to claims 18 wherein the composition further comprises a food substance or a mixture of food substances.

20. The method according to claim 19, wherein the food substance o mixture of food substances is selected from the group consisting of table margarine, shortening, mayonnaise, vegetable oil, ice cream, milk and yogurt.

21. The method according to claims 18, wherein the composition further comprises a pharmaceutically acceptable component selected from the group consisting of an excipient, antioxidant, coloring agent, binder and stabilizer.

22. The method according to claims 20 or 21, wherein the effective amount of the ester of the phytosterol or the mixture of the esters of the phytosterol of the composition comprises a daily dosage from 0.1 to 20 of said ester or said mixture of esters.

23. A composition for lowering LDL-cholesterol and triglycerides or for elevating HDL-cholesterol in blood of a mammal or both, comprising an ester of a policosanol or a mixture of esters of policosanol and an ester of a phytosterol or a mixture of esters of phytosterols wherein the acid moiety of the ester of the phytosterol or the mixture of esters of the phytosterols is a fatty acid.

24. The composition according to claim 23, wherein the acid moiety of the ester of the policosanol and the esters of the policosanols is a carboxylic acid containing from 2 to 22 carbon atoms.

25. The composition according to claims 23 or 24, wherein the fatty acid is selected from the group consisting of eicosapentaenoic acid, docosapentaenoic acid, linoleic acid, linolenic acid and arachidonic acid.

26. The composition according to claim 25, further comprising a food substance or a mixture of food substances.

27. The composition according to claim 26 wherein the food substance o mixture of food substances is selected from the group consisting of table margarine, shortening, mayonnaise, vegetable oil, ice cream, milk and yogurt.

28. The composition according to claim 25, further comprising a pharmaceutically acceptable component selected from the group consisting of an excipient, antioxidant, coloring agent, binder and stabilizer.

29. A method for lowering LDL-cholesterol and triglycerides or for elevating HDL-cholesterol in blood of a mammal or both, which comprises orally administrating to said mammal a composition containing an effective amount an ester of a policosanol or a mixture of esters of policosanols and an ester of a phytosterol or a mixture of esters of phytosterols wherein the acid moiety of the ester of the phytosterol and the mixture of esters of the phytosterols is a fatty acid.

30. The method according to claim 29, wherein the acid moiety of the esters of the policosanol esters and the mixture of esters of policosanols is a carboxylic acid containing from 2 to 22 carbon atoms.

31. The method according to claims 29 or 30, wherein the fatty acid is selected from the group consisting of eicosapentaenoic acid, docosapentaenoic acid, linoleic acid, linolenic acid and arachidonic acid.

32. The method according to claim 31, the composition further comprising a food substance or a mixture of food substances.

33. The method according to claim 32, wherein the food substance o mixture of food substances is selected from the group consisting of table margarine, shortening, mayonnaise, vegetable oil, ice cream, milk and yogurt.

34. The method according to claim 31, wherein the composition further comprise a pharmaceutically acceptable component selected from the group consisting of an excipient, antioxidant, coloring agent, binder and stabilizer.

35. The method according to claims 33 or 34, wherein the effective amount of the ester of the policosanol or the mixture of the esters of the policosanols and the esters of the phytosterol or the mixture of esters of phytosterol of the composition comprises a daily dosage from 1 to 500 mg of the ester of the policosanol or the mixture of the esters of the policosanols and 0.1 to 20 g of the ester of the phytosterol or the mixture of the esters of the phytosterols.
